(19) United States
(12) Patent Application Publication
(10) Pub. No.: US 2009/0280210 A1
(43) Pub. Date: Nov. 12, 2009

(54) Milk production method

(75) Inventors: Kwang Guan Tay, Como (AU); William John Penhale, Mount Pleasant (AU); Peter Michael Geerlings, Baldivis (AU)

Correspondence Address: EDWARDS ANGELL PALMER & DODGE LLP, P.O. BOX 55874, BOSTON, MA, 02205, US

(73) Assignee: AGRI-BIOTECH PTY LTD., Claremont, Western Australia (AU)

(21) Appl. No.: 11/665,374

(22) PCT Filed: Oct. 6, 2005
(86) PCT No.: PCT/AU05/01541

§ 371 Date: Nov. 30, 2007

(30) Foreign Application Priority Data
Oct. 6, 2004 (AU) 2004905761

(51) Int. Cl. A23C 9/152; A23C 3/00; A61D 7/00; A61K 39/395

(52) U.S. Cl. 426/2; 426/330.2; 426/231; 426/580; 424/130.1

(57) ABSTRACT

A method for improving the half-life of milk comprising the step of: a) contacting milk with an antibody raised against at least one of the following: i) a molecule required for survival by at least a micro-organism that is responsible for reducing the half-life of milk; ii) a molecule required for growth by at least a micro-organism that is responsible for reducing the half-life of milk; iii) a molecule required for survival by at least a micro-organism that aids other micro-organisms to reduce the half-life of milk; iv) a molecule required for growth by at least a micro-organism that aids other micro-organisms to reduce the half-life of milk.

### FIELD OF THE INVENTION

The present invention relates to a method improving the half-life in milk products. More particularly, it relates to a method for producing milk with an improved shelf life.

### BACKGROUND ART

Spoilage of milk products may arise from a number of sources. Milk products may spoil as a result of chemical changes caused by a reaction between the products and packaging material or contamination by foreign materials. Fats like butter are subject to rancidity caused by a chemical reaction that breaks down the fatty acids in fat to smaller molecular weight free fatty acids and, at the same time, releases certain odours.

Milk products like other foodstuffs are naturally contaminated with micro-organisms. To keep numbers of viable micro-organisms as low as possible, processes have been developed to physically remove cells—for example through improved sanitation, heating and temperature control. Refrigeration also increases the time required for milk products to spoil.

Another source of spoilage in milk products is the degradation of milk components by enzymes including lipoprotein lipases (LPLs) and metalloproteases. Enzymes are chemicals produced by all living things and bacteria produce enzymes to break down food, allowing the bacteria to access nutrients and proliferate in an environment.

Regarding LPLs, rancidity arises from the hydrolysis of milk-fat by these. LPLs can be inactivated, however some bacterial LPLs are heat resistant. Heat resistant LPLs are a major cause of rancidity (Cousin, 1982).

### SUMMARY OF THE INVENTION

In a first aspect of the invention, there is provided a method for improving the half-life in milk, processed milk products, concentrates etc. More particularly, there is provided a method for improving the shelf life of milk.

According to a first embodiment the inventive method comprises the step of: contacting milk with an antibody raised against a molecule required, for survival and or growth, by at least a micro-organism that is responsible for reducing the half-life of milk or a micro-organism that aids other micro-organisms to reduce the half-life of milk. Desirably the antibody, when contacted with the milk, will form an antibody-antigen interaction with the molecule and will deplete, remove or prevent the molecule from either being a nutrient source for the micro-organism or prevent the activity of the molecule. One such antibody is an antibody generated against lipoprotein lipase from Pseudomonas fluorescens.

According to second embodiment the method comprises the step of: inducing the production of at least an antibody in the mammary gland of an animal, which antibody is raised against a molecule required, for survival and or growth, by at least a micro-organism that is responsible for reducing the half-life of milk or a micro-organism that aids other micro-organisms to reduce the half-life of milk.

According to a third aspect the invention provides a method for the production of milk containing antibodies which method comprises the steps of: induction of antibodies according to the method detailed above and then collecting and optionally processing the antibody containing milk from the mammal. The collection of milk may be effected using normal milking processes.

According to a fourth aspect, the present invention provides a milk product with improved shelf-life, said milk product being prepared according to any one of the above methods and wherein milk produced by the methods is processed into the milk product. The invention also provides a milk product with improved shelf-life, said product being a milk product or derivative whose shelf-life is improved by the addition of an antibody preparation that binds a molecule required, for survival and or growth, by at least a micro-organism that is responsible for reducing the half-life of milk or a micro-organism that aids other micro-organisms to reduce the half-life of milk.

Other objects, features, and advantages of the instant invention, in its details as seen from the above, and from the following description of the preferred embodiment.

### BRIEF DESCRIPTION OF THE DRAWINGS

FIG. 1 shows a photograph of the supramammary gland stained blue due to migration of dye inoculated in the groin area of the animal (photo courtesy of Dr Martin CAKE, Anatomy Department Murdoch University).

FIG. 2 shows a photograph of the intranasal immunisation of a goat.

FIG. 3 shows a photograph of the implantation of an antigen releasing device in accordance with an aspect of the present invention into the groin of a sheep.

FIG. 4 shows the location of the implanted antigen releasing device of FIG. 3 in the sheep.

FIG. 5 shows a schematic diagram of the apparatus used to implant the antigen releasing device.

FIG. 6 shows a photograph of the diffusion of a lipase protein from an antigen releasing device in accordance with an aspect of the present invention into a milk agar plate. As the enzyme diffuses from the pores of the tube, it hydrolyses the lipids in the milk agar, as evident from the dark zones around the rod. The lipase protein was used as the model antigen for the development of the invention.

FIG. 7 shows a graph of the level of anti-lipase antibodies in milk from goats immunised with different protocols. The level of anti-lipase antibodies in milk from goats immunised with different protocols. The mean absorbance values of two animals were plotted over 28 days for each protocol. Maximum antibody levels were achieved with the procedure using an antigen releasing device (CRD) and intramuscular injection.

FIG. 8 shows a graph of the individual absorbance levels of anti-lipase antibodies in two separate animals implanted with an antigen releasing device (ARD) and given intramuscular injections. The levels of anti-lipase antibodies in two separate animals implanted with an antigen releasing device (CRD) and given intramuscular injections. The two results highlight the reproducible nature of the immunisation procedure

FIG. 9 shows a graph of the level of anti-lipase antibodies in serum from goats immunised with different protocols. The mean absorbance values of two animals were plotted over 28 days for each protocol.

FIG. 10 shows a comparison of mean anti-lipase anti-body levels in milk (♦) and serum (▪) produced by immunisation of goats with an antigen releasing device (ARD) and intramuscular injection.

FIG. 11 shows a graph of anti-lipase antibody production in milk (♦) and serum (▪) in goats implanted with an antigen releasing devices. The level of anti-lipase antibody levels in milk and the absence of anti-lipase antibodies in serum suggest that the antigens in the antigen releasing device implanted in the groin area are diffusing into the supramammary lymph node.

FIG. 12 shows a graph of the levels of IgG, IgA and IgM in the milk of inoculated goats. Two groups of animals were inoculated in different locations (Group 1 (G1) into the flank and Group 2 (G2) in the region adjacent to the supramammary lymph nodes) on Days 0, 10 and 19. The relative levels of IgG, IgA and IgM were determined by ELISA. The levels of all three classes of immunoglobulins were higher in the milk of Group 2 animals when compared to Group 1 animals.

FIG. 13 shows photographs of a diffusion assay of milk agar on glass slides illustrating the inhibitory effects of anti-lipase antibodies on the lipolytic activity of the lipase enzyme. Slide 1: PBS or saline was added to the wells. Slide 2: 5 mg/ml lipase from P. fluorescens. Slide 3: 5 mg/ml of lipase with an antibody negative serum (1:1 dilution). Slide 4: 5 mg/ml of lipase with serum that was positive for anti-lipase antibodies (1:1 dilution).

FIG. 14 shows photographs of a diffusion assay illustrating the inhibitory effects of anti-lipase antibodies on the lipolytic activity of the lipase enzyme on 1% milk in 1% agar. Well 1: 1:1 pre-bleed sera+PBS solution. Well 2: 1:1 pre-bled sera+lipase solution. Well 3 2.5 mg/ml lipase. Well 4 1:1 anti-lipase sera (38 days)+PBS solutions. Well 5: 1:1 anti-lipase sera (38 days)+lipase. Well 6: 0.85% PBS.

FIG. 15 shows photographs comparing the inhibitory activity of anti-lipase antibodies with a non-lipase antibodies. Antibodies to lipase were raised in the serum of two individual animals (X0247 and X0248). These anti-lipase antibody positive serum were compared to non-lipase antibody positive serum from four animals. Well 5 of all six plates contained sera containing the respective antibodies in a 1:1 dilution with the lipase enzyme.

FIG. 16 shows a graph the pH of milk incubated with lipase and eight dilutions of serum containing anti-lipase antibody, showing that as the concentrations of antibodies decreased the pH changes, suggesting a dose-response to the antibody in the serum. A positive control (lipase only with no serum) and a negative serum (saline or PBS only) were also prepared for comparison. The pH of milk was measured prior to the start of the experiment and one hour after incubation. At the 1:2 and 1:5 dilutions, the pH change was minimal. As the concentrations of antibodies decreased, the pH changes are distinct, suggesting a dose-response to the antibody in the serum.

FIG. 17 shows a graph of the pH of milk incubated with serum containing no anti-lipase antibodies. The change in pH over the incubation period (1 hr) suggests that lipase is hydrolyzing the lipids in the milk.

FIG. 18 shows a graph of the pH of milk containing anti-lipase antibodies and different levels of lipase at 37° C. for 1 hour. For the serum containing high antibody levels, there is no significant change in pH suggesting that the anti-lipase antibody inhibits the hydrolysis of lipids. In contrast, the same comparison with milk with little (or no) anti-lipase antibody shows lipid hydrolysis.

FIG. 19 shows a graph of the hydrolysis of lipids in milk by lipase (LPL) results in the decrease in pH as free fatty acids are released (♦). Serum antibodies to lipase decrease the rate of fatty acid. The rate of decrease in pH is dose dependant. The rate of decrease is greater at the lower concentration (500 μl) of antibodies (♦) when compared to the higher concentration (1000 μl) of antibodies (▴).

FIG. 20 shows a graph of the dose-dependant inhibition of the hydrolysis of lipids by serum anti-lipase antibodies. Milk was ‘spiked’ with lipase (LPL). Three different concentrations of anti-lipase antibodies were added and compared to two milk aliquots without any antibody (♦: PBS; ▪: antibody negative serum). The hydrolysis curve as measured by pH change of the lowest antibody concentration (▴: 240 μl) was similar to the tests with no antibodies, indicating that insufficient antibodies present to inhibit the enzyme. At the higher antibody concentrations (500 μl and 1000 μl), the shape and rate of hydrolysis is different, as a result of the inhibitory effect of the antibodies.

FIG. 21 shows a graph of the hydrolysis of lipids in milk in the presence and absence of anti-lipase antibodies. The inhibitory role of anti-lipase on lipase activity was assessed at 10° C. over 11 days. There is some suggestion that the anti-lipase antibody can inhibit the hydrolysis of fat (♦, ▴). However, there is evidence that other spoilage mechanisms prevail at 10° C. The rate of pH change is similar regardless; either in the presence of antibodies, in the absence of antibodies or with saline (PBS).

FIG. 22 shows a graph of the hydrolysis of lipids in milk in the presence and absence of anti-lipase antibodies. Hydrolysis of lipids by lipase in the presence and absence of anti-lipase antibodies was examined by measuring pH change at 4° C. over a 13 day interval. The pH of the antibody positive test (♦) was constant throughout the 13 day trial period. The pH of the anti-body negative test and the saline (PBS) negative control (▴ and ▪ respectively) started to decrease from about day 6. The shape of the curves differs, suggesting possible difference in the kinetics involved in milk spoilage.

### DISCLOSURE OF THE INVENTION

### General

Those skilled in the art will appreciate that the invention described herein is susceptible to variations and modifications other than those specifically described. It is to be understood that the invention includes all such variation and modifications. The invention also includes all of the steps, features, compositions and compounds referred to or indicated in the specification, individually or collectively and any and all combinations or any two or more of the steps or features.

Each document, reference, patent application or patent cited in this text is expressly incorporated herein by reference, which means that it should be read and considered by the reader as part of this text. That the document, reference, patent application or patent cited in this text is not repeated in this text is merely for reasons of conciseness.

The present invention is not to be limited in scope by the specific embodiments described herein, which are intended for the purpose of exemplification only. Functionally equivalent products, compositions and methods are clearly within the scope of the invention as described herein.

Through this specification the acronyms CRD and ARD are used interchangeably. Both refer to the antigen releasing device described, disclosed and claimed herein.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other definitions for selected terms used herein may be found within the description of the invention and apply throughout. Unless otherwise defined, all other scientific and technical terms used herein have the same meaning as commonly understood to one of ordinary skill in the art to which the invention belongs.

### DETAILED DISCLOSURE OF THE INVENTION

This invention is based on the unexpected discovery that by contacting antibodies against lipoprotein lipase enzymes produced by Pseudomonas species (e.g. Pseudomonas fluorscens) it is possible to produce a milk product with improved properties and shelf-life compared to normal pasteurised milk.

Thus, according to a first embodiment the invention provides a method for improving the half-life of milk comprising the step of:
a) contacting milk with an antibody raised against at least one of the following:
   i) a molecule required for survival by at least a micro-organism that is responsible for reducing the half-life of milk;
   ii) a molecule required for growth by at least a micro-organism that is responsible for reducing the half-life of milk;
   iii) a molecule required for survival by at least a micro-organism that aids other micro-organisms to reduce the half-life of milk;

Preferably, the method also prolongs the shelf-life of the milk.

Desirably the antibody, when contacted with the milk, will form an antibody-antigen interaction with the molecule and will deplete, remove or prevent the molecule from either being a nutrient source for the micro-organism or prevent the activity of the molecule. One such antibody is an antibody generated against lipoprotein lipase from a species of Pseudomonas such as Pseudomonas fluoroscens

The method of the present invention is performed on milk produced by mammals. Preferably, the mammals used to produce the milk are rodents or ruminants. Most preferably, the mammals are goats, sheep or cattle. Desirably the mammals are dairy cattle breeds; however dairy goat or sheep breeds may also be used.

The term “milk” used herein refers to both milk and colostrum in the form in which it is produced by the mammal or any derivative of whole milk, such as skimmed milk or whey, in liquid or in solid form.

The term “antigen” as used herein refers to any material capable of inducing an antibody response in a treated mammal, wherein the antibody is capable of binding a molecule required, for survival and or growth, by at least a micro-organism that is responsible for reducing the half-life of milk or a micro-organism that aids other micro-organisms to reduce the half-life of milk. Antigenic substances that may be employed in the invention including but not limited to: lipoprotein lipases and metalloproteinases from Pseudomonas species fluorescens (such as for example Pseudomonas). Where haptens or peptides are to be used as antigens these should first be conjugated to carrier substances such as proteins using chemistry well known to people versed in the art. (Hanly et al; Review of Polyclonal Antibody Production Procedures, ILAR Journal (1995), 37:3, 93-118).

Antibody molecules that may be used in the method of the invention include intact immunoglobulin molecules, substantially intact immunoglobulin molecules and those portions of an immunoglobulin molecule that contain the paratope. Such paratope containing portions include those portions known in the art as Fab, Fab′, F(ab′)₂ and F(v). Fab and F(ab′)₂. These portions of antibodies may be prepared by the proteolytic reaction of papain and pepsin, respectively, on substantially intact antibodies by methods that are well known. See for example, U.S. Pat. No. 4,342,566. Fab′ antibody portions are also well known and are produced from F(ab′)₂ portions followed by reduction of the disulfide bonds linking the two heavy reduction of the disulfide bonds linking the two heavy chain portions as with mercaptoethanol, and followed by alkylation of the resulting protein mercaptan with a reagent such as iodoacetamide. An antibody containing intact antibody molecules are preferred, and are utilised as illustrated herein.

In a second aspect of the invention, there is provided a method for improving the half-life of milk comprising the step of:
a) inducing the production of at least an antibody in the mammary gland of an animal, which antibody is raised against at least one of the following:
   i) a molecule required for survival by at least a micro-organism that is responsible for reducing the half-life of milk;
   ii) a molecule required for growth by at least a micro-organism that is responsible for reducing the half-life of milk;
   iii) a molecule required for survival by at least a micro-organism that aids other micro-organisms to reduce the half-life of milk;
   iv) a molecule required for growth by at least a micro-organism that aids other micro-organisms to reduce the half-life of milk.

Preferably, the method also prolongs the shelf-life of the milk.

Methods for the induction of antibody production in milk will include the step of: implanting at least one antigen releasing device adjacent to or within at least one supramammary lymph node, wherein in use the antigen releasing device releases an antigen into the tissue area around the supramammary lymph node which stimulates antibody secretion into a mammary gland.

According to this aspect of the invention the distance of the implant from the supramammary gland should be at least close enough that the release of antigen from the antigen releasing device causes the antibody response of the mammal (into which it is implanted) to be maintained at a level facilitates the production of antibodies in milk at levels that are therapeutically or anti-microbially suitable. For example, the ARD may be implanted in the udder. Alternatively by way of illustration the antigen releasing device is preferably implanted at a distance of up to 150 mm from at least one supramammary lymph node, wherein in use the antigen releasing device releases an antigen into the tissue area around the supramammary lymph node which stimulates antibody secretion into a mammary gland. Preferably, the antigen releasing device is implanted either adjacent to or at a distance of between about 1 mm and 100 mm from the supramammary lymph node. Most preferably, the distance is between about 50 mm and 100 mm.

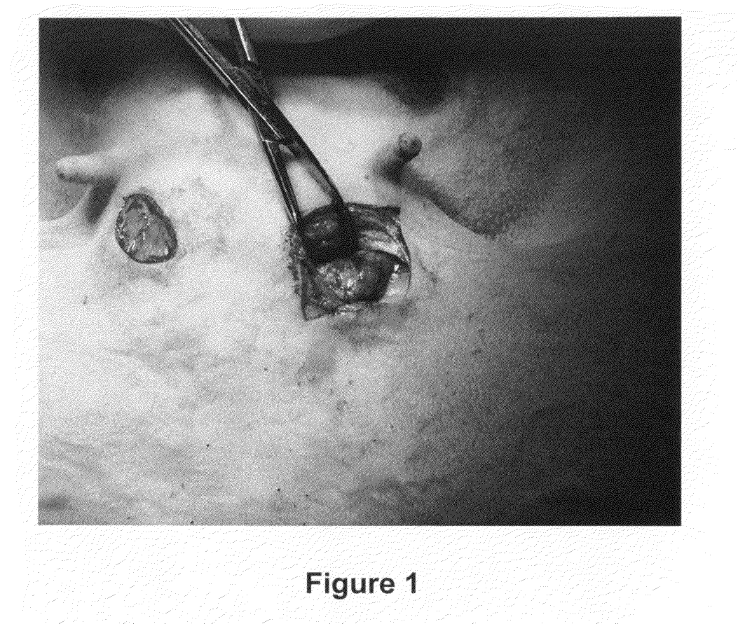

Implantation of the antigen releasing device adjacent to or within at least one supramammary lymph node causes the antigen contained in the antigen releasing device to be released into the tissue near and within the node (FIG. 1). This in turn stimulates the node to generate antibodies to the antigen. These antibodies are secreted into the mammary glands and therefore enter the milk of the mammal.

The size, characteristics and choice of antigen releasing device, is dependant on the size and properties of the antigen of interest. It is desirable that the choice of antigen releasing device allows the antigen contained therein to be released from the device at a rate which causes the antibody response of the mammal into which it is implanted to be maintained at a desirable level.

Devices for slow release of compositions are described in, for example, U.S. Pat. No. 3,279,996, whilst immunopotentiating devices for the sustained release of antigen are described, for example, in Australian Patent No. 740133

A porous silicon implant impregnated with a beneficial substance is described in Patent No. DE69917625D. An implantable device for molecule delivery is described in U.S. Pat. No. 6,716,208. Other suitable examples of sustained-release preparations include semipermeable matrices of solid hydrophobic polymers containing the protein, which matrices are in the form of shaped articles, e.g., films, or microcapsules compressed into delivery devices. Examples of sustained-release matrices include polyesters, hydrogels (e.g., poly(2-hydroxyethyl-methacrylate) as described by Langer et al., J. Biomed. Mater. Res. 15:167-277 (1981) and Langer, Chem. Tech. 12:98-105 (1982) or poly(vinylalcohol)], polylactides (U.S. Pat. No. 3,773,919, EP 58,481), copolymers of L-glutamic acid and gamma ethyl-L-glutamate (Sidman et al., Biopolymers 22:547-556 [1983]), non-degradable ethylene-vinyl acetate (Langer et al., supra), degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid (EP 133,988). Another reference is B. Baras, M. A. Benoit & J. Gillard (2000) “Parameters influencing the antigen release from spray dried poly (DL-lactide) microparticles.” International Journal of Pharmaceutics, 200:133-145.

While polymers such as ethylene-vinyl acetate and lactic acid-glycolic acid enable release of molecules for over 100 days, certain hydrogels release proteins for shorter time periods. When encapsulated antigens remain in the body for a long time, they may denature or aggregate as a result of exposure to moisture at 37° C., resulting in a loss of biological activity and possible changes in immunogenicity. Rational strategies can be devised for antibody stabilization depending on the mechanism involved. For example, if the aggregation mechanism is discovered to be intermolecular SS or disulfide bond formation through thio-disulfide interchange, stabilisation may be achieved by modifying sulfhydryl residues, lyophilising from acidic solutions, controlling moisture content, using appropriate additives, and developing specific polymer matrix compositions.

Sustained-release fragment compositions also include liposomally entrapped fragments. Liposomes containing the antibody are prepared by methods known per se: DE 3,218,121; Epstein et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; Japanese patent application 83-118008; U.S. Pat. Nos. 4,485,045 and 4,544,545; and EP 102,324. Ordinarily the liposomes are of the small (about 200-800 Angstroms) unilamellar type. Other devices for the slow release of antigen into the tissue near the supramammary lymph node are encompassed within the present invention.

An effective amount of antigen to be employed therapeutically will depend, for example, upon the objectives, the route of administration, the type of antigen and/or adjuvant and the condition of the mammal. Accordingly, it will be necessary for the therapist to titre the dosage and modify the mode of administration as required to obtain the optimal effect. Typically, the operator will administer an antigen until a dosage is reached that achieves the desired effect. The progress of this therapy is easily monitored by conventional assays.

In the further aspect of the invention, there is provided a method for inducing the sustained release of antibodies in milk comprising the further step of administering a primer composition by an administration route selected from intramammary, intraperitoneal, intramuscular or intranasal. Administration of a primer may take place before, during or after implanting the antigen releasing device. It is preferable that the primer composition be delivered to a mucosal surface so that antibody production on mucosal surfaces (of which the mammary gland is one) is preferentially stimulated.

The primer composition administration could be a single administration, or could comprise a number of administrations at intervals over a period of days or weeks. Timing of the administration of primer composition is generally spaced based on contemporary immunisation protocols (for example, every 2 weeks). To avoid local irritation and congestion, it is usually preferred that the primer composition not be administered to the same site more frequently than every second week. The initial exposure of this priming step stimulates the low level production of antibodies, which production is then increased and maintained by antigen released by the antigen releasing device.

The method of the present invention may also comprise the additional step of administering a booster composition comprising antigen to a mammal by an administration route selected from intramammary, intraperitoneal, intramuscular and/or intranasal after the antigen releasing device has been implanted. It is preferable that the booster composition be delivered to a mucosal surface so that antibody production on mucosal surfaces (of which the mammary gland is one) is preferentially stimulated.

Such booster compositions could be administered as a single administration, or could comprise a number of administrations at intervals over a period of days or weeks. Administration of booster compositions is generally spaced to suit the convenience of the operator. To avoid local irritation and congestion, it is usually preferred that administration of the booster composition to the same site not be more frequent than every other week.

In a preferred method, the antigen administered is the same for each step of the method. Therefore, the same antigen may be used in the antigen releasing device, the primer composition and/or the booster composition.

The use of adjuvants both within the antigen releasing device and in the compositions used for priming and boosting is also desirable. An adjuvant can serve as a tissue depot that slowly releases the immunogen and also as a lymphoid system activator that non-specifically enhances the immune response [Hood et al., in Immunology, p. 384, Second Ed., Benjamin/Cummings, Menlo Park, Calif. (1984)]. Suitable adjuvants for use with the antigens of the invention include but are not limited to the following: Freund's complete adjuvant (FCA), Freund's incomplete adjuvant (FIC), TiterMax Gold™, adjuvant 65, cholera toxin B subunit, IL1-B Fragment 163-171 synthetic human adjuvant, alhydrogel; or bordetella pertussis, muramyl dipeptide, cytokines, saponin, Adju-Phos, Algal Glucau, Algammuliu, Alhydrogel, Antigen. Formulation, Avridine, Bay R1005, calcitrial, calcium phosphate, Gel, CRL 1005, cholera Holotoxin (CT), DDA, DHEA, DMPC, DMPG, DOC/Alum Complex, Gamma Inulin, Gerbu Adjuvant, GMDP, Imiquimod, Imuither, Interferon-gamma, ISCOM(s), Iscoprop 7.0.3, Loxoribine, LT-OA or LT Oral adjuvant, MF59, MONTANIDE ISA51 and ISA720, MPL, MTP-PE, MTP PE Liposomas, murametide, murapaimitive, NAGO, Nonionic surfactant vesides, Pleuram, PLGA, PGA and PLA, Pluronic L121, PMMA, PODDS, Poly Ra, Polyru Polyphophazene, Polysorbate 80, Protein Cochleates, QS-21, Quil A, Rehydrogel HPA, Rehydrogel LV, S-28465, SAF-1, Sclavo, peptide, Seudai Protediposomes, sendai-containing lipid matrices, Span 85, special, squalene, stearyl Tyrosine, Theramide, Threonyl-MDP, Ty Particles, saponin Q521, MF59, Alum.

In relation to the step of administering the priming composition or the boosting composition, preferably the antigenic substances are suspended in liquid medium for infusion or injection according to known protocols. Any appropriate carriers, diluents, buffers, and adjuvants known in the art may be used. Suitable suspension liquids include saline solution, water, and physiologic buffers.

If administration of the priming composition or the boosting composition is by injection, preferably prior to injection the antigens are emulsified in appropriate carriers with adjuvant using, for example, a laboratory homogeniser. In one example of such a method, aqueous antigen is mixed with 3 volumes of adjuvant and emulsified until a stable water-in-oil emulsion is formed. The presence of a stable emulsion can be demonstrated using tests well known in the art.

In a further aspect of the invention, the method further comprises a preselection step. In this step individual animals are tested and selected for their ability to produce antibodies. Considerable between-animal variability exists for the production of antibodies. This preselection step, wherein the animals showing the best antibody titre responses are selected, assists in decreasing the between-animal variability factor. This process may similarly be used to build groups of animals particularly suited to antibody production.

According to a third aspect the invention provides a method for the production of milk with a prolonged half-life containing antibodies, which method comprises the steps of: induction of antibodies according to the method detailed above and then collecting and optionally processing the antibody containing milk from the mammal. The collection of milk may be effected using normal milking processes. Preferably, the method also prolongs the shelf-life of the milk.

According to a fourth aspect, the present invention provides a milk product with improved half-life, said milk product being prepared from milk generated according to any one of the above methods and wherein milk produced by the methods is processed into the milk product.

The invention also provides a milk product with improved half-life, said product being a milk product or derivative whose half-life is improved by the addition of an antibody raised against at least one of the following:
i) a molecule required for survival by at least a micro-organism that is responsible for reducing the half-life of milk;
ii) a molecule required for growth by at least a micro-organism that is responsible for reducing the half-life of milk;
iii) a molecule required for survival by at least a micro-organism that aids other micro-organisms to reduce the half-life of milk;
iv) a molecule required for growth by at least a micro-organism that aids other micro-organisms to reduce the half-life of milk.

This milk is useful in the form obtained directly from the mammal but may be processed if required. Examples of processing steps include heat treatment, ultra violet radiation, concentration, supplementation with food additives, drying into concentrates, milk powders and the like.

The present invention also provides a milk product with prolonged shelf-life, said product being a milk product or derivative whose shelf-life is prolonged by the addition of an antibody raised against at least one of the following:
i) a molecule required for survival by at least a micro-organism that is responsible for reducing the shelf-life of milk;
ii) a molecule required for growth by at least a micro-organism that is responsible for reducing the shelf-life of milk;
iii) a molecule required for survival by at least a micro-organism that aids other micro-organisms to reduce the shelf-life of milk;
iv) a molecule required for growth by at least a micro-organism that aids other micro-organisms to reduce the shelf-life of milk.

Milk and milk products in which the method of the invention may have benefit may include foodstuffs, drinks (e.g., energy or sports drinks) and animal feeds. For example, the milk may possibly be used as an ingredient in, baby food, bakery products (for example, a bread, yeasted good or cake) or bakery supply products (for example, custard or bakery fillings or toppings), batter or breading, cereal, confectionary, flavour or beverage emulsions, fruit fillings, gravy, soups, sauces (eg meat sauces) or food thickeners, UHT treated gravy, meal components (e.g., vegetarian meal/components), meat products (e.g., comminuted meat products, sausages, burgers, grill steaks, canned meats, meat pies, fish preparations, meat patties, meat spread and pastes), pizza toppings, pet foods, pharmaceuticals or neutraceuticals, potato products, dressings (e.g., salad or low fat dressings), snack or cracker spreads (e.g., savoury or sweet spreads), pasta products (e.g. noodles), fat-filled powders, quiches or flans, cheese or cream mimetics and other substitute dairy products not specifically detailed within this description of invention (e.g. desserts, flavoured milk drinks, milk shakes, cheeses, cheese spreads or dips).

### EXAMPLES

Further features of the present invention are more fully described in the following non-limiting Examples. It is to be understood, however, that this detailed description is included solely for the purposes of exemplifying the present invention. It should not be understood in any way as a restriction on the broad description of the invention as set out above.

### Example 1

### Preparation of Antigen Releasing Device Minus Adjuvant

### Materials

(i) 0.15 g mannitol-D (Sigma-Aldrich)
(ii) 0.15 g sodium citrate (Proanalys)
(iii) 11.25 mg lipase from Pseudomonas fluorescens (Sigma-Aldrich)
(iv) 0.75 ml part A Silastic (Dow Corning Q7-4850)
(v) 0.75 ml part B Silastic (Dow Corning Q7-4850)
(vi) 2×2.5 ml disposable syringes (Terumo)
(vii) 2×1 ml syringe (Terumo)
(viii) 2×12 G×4 inch stainless steel hypodermic needles
(ix) 37° C. incubator
(x) sterile petri dish
(xi) sterile scalpel
(xii) sterile spachella
(xiii) 32 ml glass McCartney bottle

### Methods

(i) Remove pistons from all syringes
(ii) Part A—silastic placed into 1 ml syringe using spatula. Piston replaced into syringe and 0.75 ml quantity dispensed into one 2.5 ml syringe.
(iii) Procedure repeated for Part B.
(iv) Lipase, mannitol and sodium citrate are combined and mixed in a small glass McCartney then placed into the 2.5 ml syringe containing Part A of the silastic. Part B was then expelled from its syringe into the other 2.5 ml syringe effectively ‘sandwiching’ the lipase, mannitol and sodium citrate between Part A and Part B of the silastic in one syringe. The piston was replaced in this syringe and removed from the empty syringe. The contents of the first syringe were expelled into the second syringe then its piston replaced. The procedure was repeated 20 times to effectively mix all reagents. The reagents were finally expelled into two 12 G needles and stored at 37° C. for three days. The cured silastic was extracted from the needles, cut into 3 cm lengths in the open petri dish and placed under UV light for 24 hours before being stored in sterile 10 ml centrifuge tubes at −20° C. Each 3 cm ARD contained 1 mg lipase, 13 mg mannitol, 13 mg sodium citrate, in 250 μl of total silastic.

### Example 2

### Preparation of Antigen Releasing Device Including Adjuvant

### Materials

(i) 0.3 g mannitol-D (Sigma-Aldrich)
(ii) 0.3 g sodium citrate (Proanalys)
(iii) 22.50 mg lipase from Pseudomonas fluorescens (Sigma-Aldrich)
(iv) 1.2 mg IL1-B Fragment 163-171 synthetic human (Sigma)

### (v) 1.5 ml Part A Silastic (Dow Corning Q7-4850)

### (vi) 1.5 ml Part B Silastic (Dow Corning Q7-4850)

(vii) 2×2.5 ml disposable syringes (Terumo)
(viii) 2×1 ml syringe (Terumo)
(ix) 2×12 G×4 inch stainless steel hypodermic needles
(x) 37° C. incubator
(xi) sterile petri dish
(xii) sterile scalpel
(xiii) sterile spachella
(xiv) 32 ml glass McCartney bottle

### Methods

(i) Remove pistons from all syringes
(ii) Part A silastic placed into 1 ml syringe using spatula. Piston replaced into syringe and 0.75 ml quantity dispensed into one 2.5 ml syringe.
(iii) Procedure repeated for Part B.
(iv) Lipase, mannitol and sodium citrate mixed in a small glass McCartney then placed into the 2.5 ml syringe containing Part A of the silastic. Part B was then expelled from its syringe into the other 2.5 ml syringe effectively ‘sandwiching’ the lipase, mannitol and sodium citrate between Part A and Part B of the silastic in one syringe. The piston was replaced in this syringe and removed from the empty syringe. The contents of the first syringe was expelled into the second syringe then its piston replaced. The procedure was repeated 20 times to effectively mix all reagents. The reagents were finally expelled into two 12 G needles and stored at 37° C. for three days. The cured silastic was extracted from the needles, cut into 3 cm lengths in the open petri dish and placed under UV light for 24 hours before being stored in sterile 10 ml centrifuge tubes at −20° C. Each 3 cm ARD contained 1 mg lipase, 13 mg mannitol, 13 mg sodium citrate, 50 μg IL1-B, in 250 μl of total silastic.

### Example 3

### Delivery of ARD

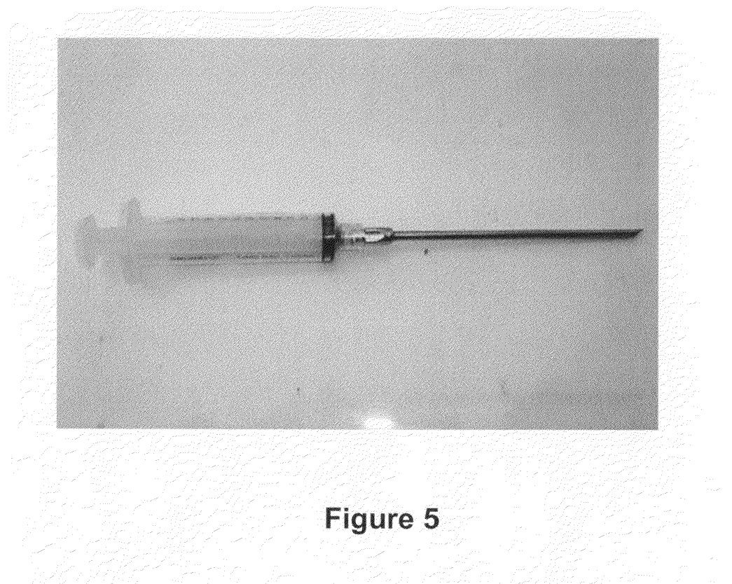

A device was purpose designed and built comprising a 10 ml disposable luer lock syringe, a 10 G×4 inch stainless steel hypodermic needle and a 90 mm×10 G stainless steel welding rod (see FIG. 5). The device was assembled with the rod attached to the piston of the syringe and passing through the needle. The piston was withdrawn about 3 cm allowing for free space near the tip of the needle in which to insert the antigen releasing device. Thus, when the piston is depressed, the antigen releasing device is expelled from the needle by the rod (see FIG. 5).

The animal was placed on the floor on its back and restrained by animal handlers. An area approximately 3 cm×10 cm right lateral and adjacent to the udder was swabbed with iodine. 2 ml of 2% lignocaine infiltrated the cutaneous tissue through a 26 G hypodermic needle as a local anaesthetic. The 10 G needle housing the ARD was inserted in the posterior end of this area and pushed to the anterior end subcutaneously. The piston was depressed and needle withdrawn simultaneously. The point of insertion was swabbed with iodine. In most cases the antigen releasing device could be felt in situ.

### Example 4

### Preparation of Lipase Nasal Inoculum

### Materials

(i) 15.5 mg lipase from P. fluorescens (Sigma-Aldrich)
(ii) 38.75 ml 0.85% saline (Excel laboratories)
(iii) 1 mg Cholera Toxin B Subunit (US Biological)
(iv) 2.5 ml syringes
(v) 50 ml sterile tube (Falcon)
(vi) 8-10 cm length of approximately 20 G plastic tube (e.g. from winged infusion set (Terumo))

### Methods

(i) All reagents were combined in 50 ml tube on day of first administration.
(ii) The remainder was stored at 4° C. until used.

### Example 5

### Delivery of Nasal Inoculum

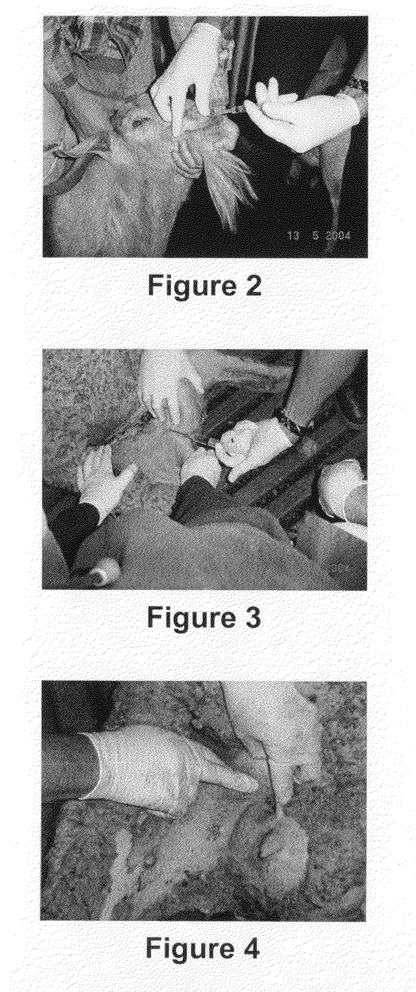

2.5 ml of inoculum (containing 1 mg lipase, 64.5 μg Cholera Toxin B Subunit) was drawn into syringe with the 20 G tube from the infusion set attached. 60-80% of tube length was placed into right nostril of animal and slowly dispensed whilst simultaneously withdrawing the tube from the nostril (FIG. 2).

### Example 6

### Preparation of Lipase Intramuscular Injected Inoculum

### Materials

(i) 36.5 mg of lipase from P. fluorescens (Sigma)
(ii) 9.1 ml of 0.85% saline (Excel Laboratories)
(iii) TiterMax Gold™ adjuvant
(iv) glass McCartney bottle
(v) 3 ml all plastic syringes (Teruma)
(vi) stainless steel double-hub
(vii) 23 G×1 inch hypodermic needle

### Methods

(i) Lipase and saline were combined in glass McCartney giving a concentration of 4 mg/ml on the day of first administration. The remainder was stored at 4° C. until required.

### Example 7

### Delivery of Intramuscular Inoculum

0.5 ml of 4 mg/ml lipase in saline solution as drawn into syringe and emulsified with 0.5 ml TiterMax Gold™ as per manufacturers instructions. Inoculum was administered intramuscularly (IM) in the rear of the left hind leg using 23 G×1 inch needle attached to the syringe. Animals received IM injections Day 7, Pay 14 and Day 21.

### Example 8

### Immunisation Protocol 1

The animals were given an IM injection in the flank, the commonly used route for immunisation.

Two animals were immunised by the intramuscular route (IM) with lipase from Pseudomonas fluorescens on day 0 with 1250 μg lipase emulsified with 750 ul titremax. The animals received a primary boost (950 ug lipase and 560 ul titremax) and a secondary boost (2500 ug lipase and 500 μl titremax) on day 12 and day 24 respectively. Animals were bled on the days of immunisation and blood samples were collected at monthly intervals after the immunisation program.

### Example 9

### Immunization Protocol 2

The animals were given antigen inoculation by antigen releasing device and/or injection. Table 1 summarises the schedule of inoculums and refers to 6 animals and one antigen.

TABLE 1
Protocols for initial trial of antigen releasing device (ARD).
Trial CRD and S/C injection of Ag to assess Ab secretion via mammary

| Date | Tag | Ag | Amount | Adjuvant | Amount | Delivery | Total Vol | Location | Milk y/n | Bleed y/n |
|---|---|---|---|---|---|---|---|---|---|---|
| 28 Apr. 2004 | White 651 8RS | Lipase | 2.5 mg | T/Max | 500 ul | S/C | 1 ml | groin | Y | Y |
| 28 Apr. 2004 | Black 1292 BX8 | Lipase | 2.5 mg | T/Max | 500 ul | S/C | 1 ml | groin | Y | Y |
| 28 Apr. 2004 | Black 1675 BX8 | Lipase | 250 ug unsheathed silastic CRD | | | S/C | | RH groin | Y | Y |
| 28 Apr. 2004 | White 552 8RS | Lipase | 250 ug sheathed silastic CRD | | | S/C | | RH groin | Y | Y |
| 13 May 2004 | White 651 8RS | Lipase | 0 | 0 | 0 | 0 | 0 | 0 | Y | Y |
| 13 May 2004 | Black 1292 BX8 | Lipase | 0 | 0 | 0 | 0 | 0 | 0 | Y | Y |
| 13 May 2004 | Black 1675 BX8 | Lipase | | CRD | | | | | Y | Y |
| 13 May 2004 | White 552 8RS | Lipase | | CRD | | | | | Y | Y |
| 13 May 2004 | White 367 | lipase | 1 mg/12 mg mannitol/12 mg SC-CRD | | | | 30 × 2 mm | Rh groin | Y | Y |
| 13 May 2004 | Orange 468 | lipase | 1 mg/12 mg mannitol/12 mg SC-CRD | | | | 30 × 2 mm | Rh groin | Y | Y |
| 28 May 2004 | White 651 8RS | Lipase | 2.5 mg | T/Max | 500 ul | S/C | 1 ml | RH rump | Y | Y |
| 28 May 2004 | Black 1292 BX8 | Lipase | 0 | 0 | 0 | 0 | 0 | 0 | Y | Y |
| 28 May 2004 | Black 1675 BX8 | Lipase | 0 | 0 | 0 | 0 | 0 | 0 | Y | Y |
| 28 May 2004 | White 552 8RS | Lipase | 2.5 mg | T/Max | 500 ul | S/C | 1 ml | RH rump | Y | Y |
| 28 May 2004 | White 367 | lipase | 2.5 mg in 2.5 ml saline | | | | 2.5 ml | nasal | Y | Y |
| 28 May 2004 | Orange 468 | lipase | 2.5 mg | T/Max | 500 ul | S/C | 1 ml | Lhrump | Y | Y |

| Date | Tag | Ag | Milk y/n | Bleed y/n |
|---|---|---|---|---|
| 1 Jul. 2004 | White 367 | lipase | Y | Y |
| 1 Jul. 2004 | Orange 468 | lipase | Y | Y |

### Example 10

### Immunisation Protocol 3

Protocols used 2 animals per group with 12 goats used in total. Six different protocols were evaluated in two separate animals to determine an optimal procedure to produce sustained antibody levels in milk, summarised in Table 4 and 5.

TABLE 4
The immunisation protocols used to stimulate the production of antibodies in milk.

| | | | | | | | Sampling Frequency | |
|---|---|---|---|---|---|---|---|---|
| | | | Innoculatio Protocol | | | | Milk | Blood |
| Group | Description | Adjuvant | Day 1 | Day 7 | Day 14 | Day 21 | Collection | Collection |
| 1 | Intramuscular (IM) | YES | | IM Injection | IM boost | IM injection | Daily | 3-4 day |
| | Injection only | | | | | | | intervals |
| 2 | ARD only | YES | Implant ARD | | | | Daily | 3-4 day |
| | | | | | | | | intervals |
| 3 | Intranasal (IN) spray | YES | | IN Innoculati [symbol] | IN boost | IN boost | Daily | 3-4 day |
| | only | | | | | | | intervals |
| 4 | Intramuscular (IM) | YES | Implant ARD | | | | Daily | 3-4 day |
| | injection + ARD | | | | | | | intervals |
| 5 | Intranasl (IN) spriay + | YES | | IM Injection | IM boost | IM injection | Daily | 3-4 day |
| | ARD | | | | | | | intervals |
| 6 | ARD only | NO | Implant ARD | | | | Daily | 3-4 day |
| | | | | | | | | intervals |

[symbol]

indicates data missing or illegible when filed

TABLE 5
The immunisation protocols used to stimulate
the production of antibodies in milk.

| | | | | | |
|---|---|---|---|---|---|
| S/Mark | Inoculum | Amount | Delivery | Site | Freq |

Group 1- inject

| | | | | | |
|---|---|---|---|---|---|
| lipase | 2.0 | mg | I.M | R/h quart | Day 7 |
| TiterMax | 500 | ul | | | Day 14 |
| saline | 500 | ul | | | Day 21 |

Group 2 - CRD inc Adj

| | | | | | |
|---|---|---|---|---|---|
| lipase | 1 | mg | CRD | RH groin | Day 0 |
| mannitol | 13 | mg | | | |
| sod citrate | 13 | mg | | | |
| IL-1B | 50 | ug | | | |
| silastic | 250 | ul | | | |

Group 3 - nasal only

| | | | | | |
|---|---|---|---|---|---|
| lipase | 1 | mg | nasal | nostril | Day 7 |
| C/Tox | | | | | Day 14 |
| saline | 2.5 | ml | | | Day 21 |

Group 4 - CRD & inj

| | | | | | |
|---|---|---|---|---|---|
| lipase | 1 | mg | CRD | RH groin | Day 0 |
| mannitol | 13 | mg | | | |
| sod citrate | 13 | mg | | | |
| IL-1B | 50 | ug | | | |
| silastic | 250 | ul | | | |
| lipase | 2.0 | mg | I.M | R/h quart | Day 7 |
| TiterMax | 500 | ul | | | Day 14 |
| saline | 500 | ul | | | Day 21 |

Group 5 - CRD & nasal

| | | | | | |
|---|---|---|---|---|---|
| lipase | 1 | mg | CRD | RH groin | Day 0 |
| mannitol | 13 | mg | | | |
| sod citrate | 13 | mg | | | |
| IL-1B | 50 | ug | | | |
| silastic | 250 | ul | | | |
| lipase | 1 | mg | nasal | nostril | Day 7 |
| C/Tox | | | | | Day 14 |
| saline | 2.5 | ml | | | Day 21 |

Group 6 - CRD minus Adj

| | | | | | |
|---|---|---|---|---|---|
| lipase | 1 | mg | CRD | RH groin | Day 0 |
| mannitol | 13 | mg | | | |
| sod citrate | 13 | mg | | | |
| silastic | 250 | ul | | | |

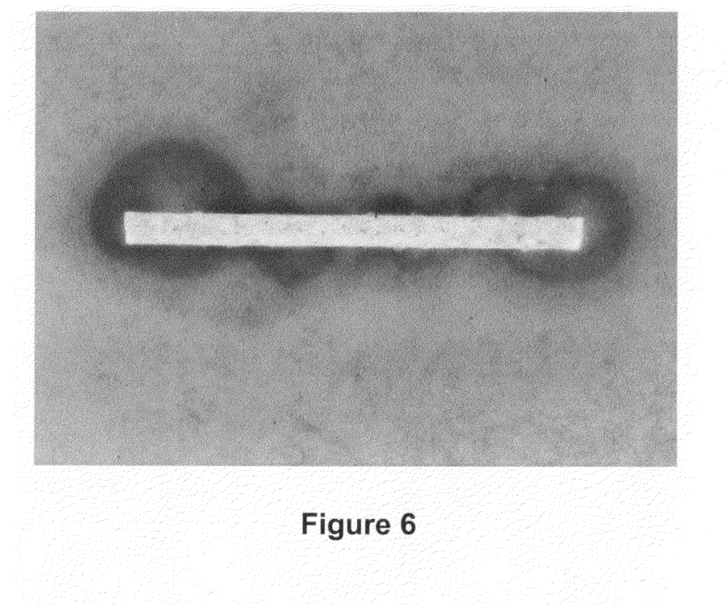

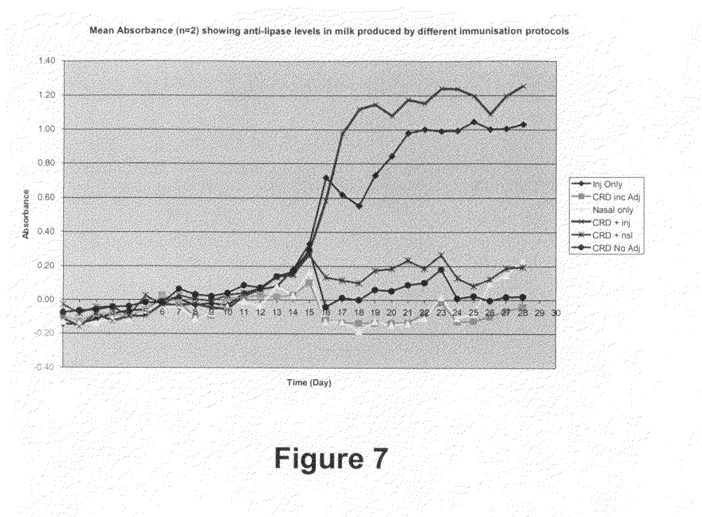

A total of 12 goats were studied, with two goats dedicated to each inoculation regime. The presence of anti-lipase antibodies was evaluated with Enzyme-Linked Immunosorbent Assay (ELISA). The mean absorbance value less that of the blank control of the daily milk samples were plotted (FIG. 6). Results were reproducible between animals, as shown in FIG. 7.

All six regimens were successful in raising antibodies. However, the relative concentrations of antibodies for each group varied. The highest mean absorbance value; which is indicative of the greatest concentration of antibodies produced; was recorded for the Group 4 animals. The Group 4 animals received an ARD implant on Day 0 of the program and 3 subsequent injections to the back flank area on Days 7, 14 and 21.

The mean absorbance value of Group 4 was greater than the value produced by the Group 1 goats, who only received IM injections at day 7, 14 and 21.

Both Group 1 and 4 animals showed some response up to approximately Day 14. At Day 15, the levels of anti-lipase antibodies increased substantially, presumably a consequence of the secondary immune response. The higher levels of antibodies were sustained for the duration of the study, in this case up to Day 28.

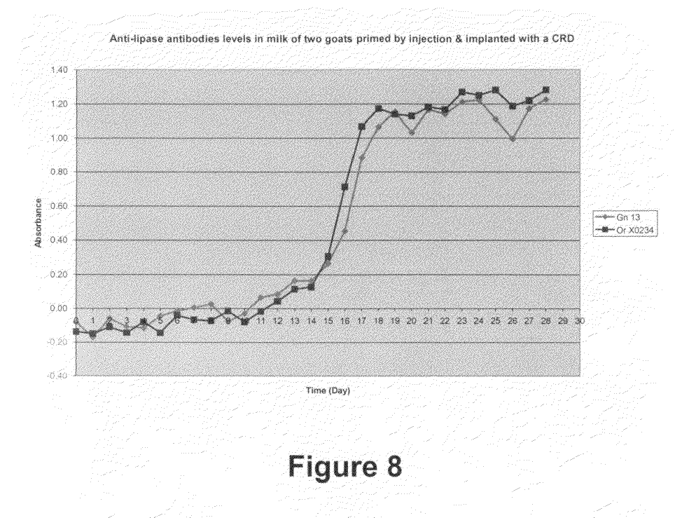

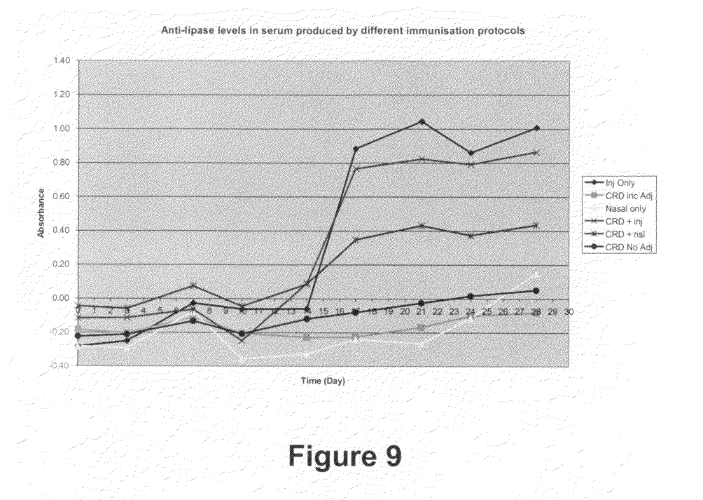

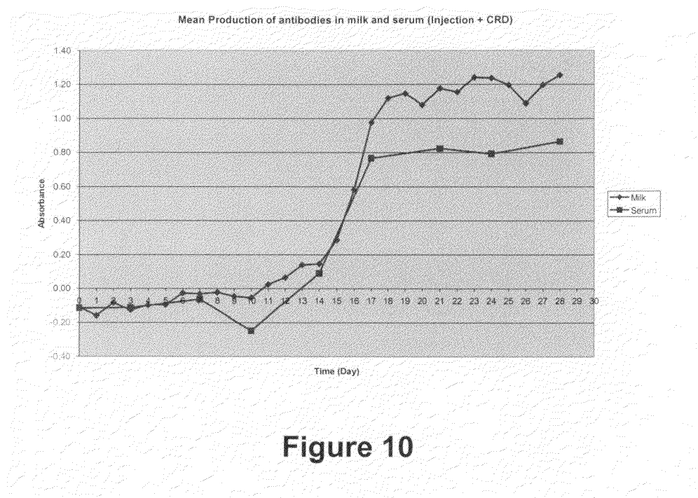

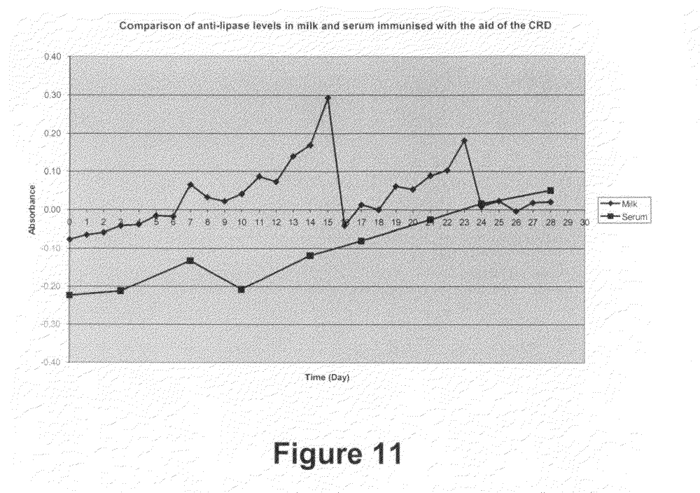

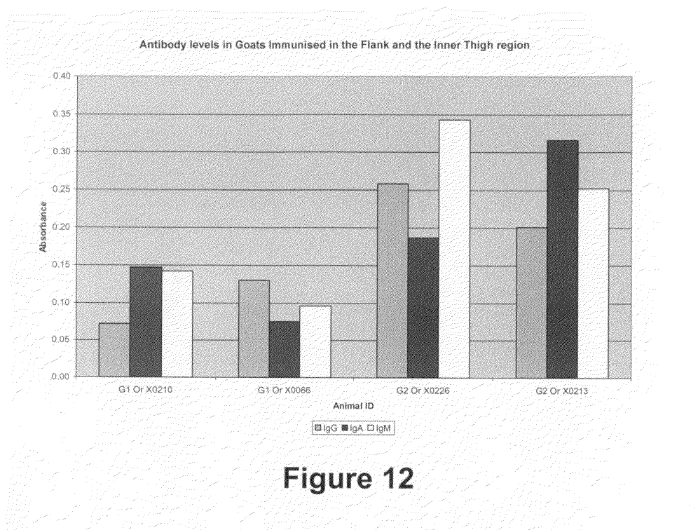

The levels of anti-lipase antibodies in the serum of the inoculated animals was also measured, as shown in FIGS. 8, 9, 10 and 11. FIG. 8 shows results of the individual absorbance levels of anti-lipase antibodies in two separate animals implanted with an antigen releasing device (ARD) and given intramuscular injections. FIG. 9 shows results of the level of anti-lipase antibodies in serum from goats immunised with different protocols. FIG. 10 shows results of a comparison of mean anti-lipase anti-body levels in milk and serum produced by immunisation of goats with an antigen releasing device (ARD) and intramuscular injection. FIG. 11 shows the anti-lipase antibody production in milk and serum in goats implanted with an antigen releasing device. The level of anti-lipase antibody levels in milk and the absence of anti-lipase antibodies in serum suggest that the antigens in the antigen releasing device implanted in the groin area are diffusing into the supramammary lymph node.

### Example 11

### Immunization Protocol 4

The protocol used two lactating goats (Capra hircus) for each group, with four goats used in total. The antigen used was lipase from Pseudomonas fluorescens.

Preparation for Immunisation Protocol
1. Emulsify 30 mg of lipase in 15 ml 0.85% saline.
2. Emulsification with Titermax Gold was according to manufacturer's, specification. The lipase in saline solution was mixed with an equal volume of Titremax Gold (1:1).
3. Dispense 1 ml into 2.5 ml syringe for administration (each 1 ml dose contains 1 mg lipase)
4. Two groups of animals were inoculated on Day 0, 10 and 19.
5. Group 1 was inoculated in the left flank and Group 2 was inoculated adjacent to the supramammary lymph node.
6. Milk and serum was collected.

Coating plates for Enzyme Linked Immunosorbent Assay (ELISA)
1. Prepare 2.5 μg/ml of antigen in coating buffer.
2. 100 μl of the mixture was dispensed into each well of a 96 well ELISA tray.
3. The plates were covered and were left to stand overnight at 4° C.

Enzyme Linked Immunosorbent Assay (ELISA) Protocol
1. Prior to use, the coated plates were washed 3 times with PBS Tween.
2. A serum diluent of 1% Human serum in PBS Tween.
3. Load 100 μl of the serum diluent into each well.
4. Load 1 μl of sample of interest to well.
5. Plates were incubated at 37° C. for 2 hours.
6. Plates were washed 3 times with PBS Tween.
7. 1/1000 dilutions of mouse α-sheep IgG, mouse α-sheep IgA and mouse α-sheep IgM in 1% serum diluent (1% Human serum in PBS Tween) were prepared.
8. Load 100 μl into respective wells.
9. The plates were placed in 37° C. for 2 hrs.
10. The plates were washed 3 times with PBS Tween.
11. 1/2500 dilution of rabbit α-mouse IgG (H+L) in 1% serum diluent was prepared.
12. Load 100 μl per well.
13. Incubate at 37° C. for 2 hrs.
14. The plates were washed 3 times with PBS Tween.
15. A 1/100 dilution of 250 mg/ml Nitrophenyl phosphate in Diethanolamine Buffer was prepared.
16. Load 100 μl per well.
17. Incubate at room temperature for approximately 20 to 30 minutes.
18. The reaction was terminated with 50 ul of 3.75M NaOH.
19. ELISA plates were read at 405 nm.

Results

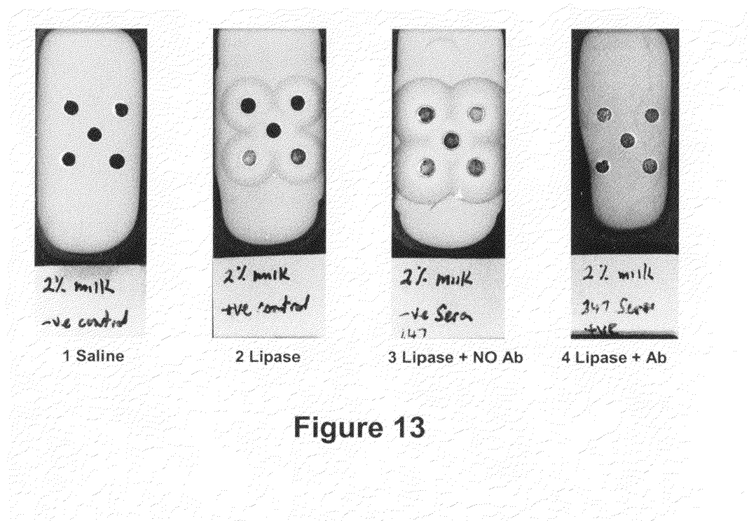

Milk collected was analysed by ELISA for levels of IgG, IgA and IgM. Results from Group 1 (intermuscular into the flank—designated G1) and Group 2 (stimulation of the supramammary lymph node—G2 animals) in FIG. 13 shows higher levels of all three classes of immunoglobulin produced in the milk of Group

### Example 12

### Collection and Storage of Milk Samples

### Materials

(i) Beckman Acuspin refrigerated centrifuge
(ii) Rennet Type II from Mucor meihei (Sigma) in Hp water at a concentration of 2 mg/ml
(ii) 10 ml sterile centrifuge tubes
(iv) P1000 pipetteman
(v) 1 ml disposable transfer pipettes
(vi) 5 ml plastic storage tubes
(vii) 37° C. incubator

### Method

Milk was collected by hand milking into 32 ml glass McCartney bottles in the absence of any chemical or mechanical stimuli. Milk was generally collected in the morning without prior separation from kids. Samples were stored on ice after collection and transferred as soon as practicable thereafter. Milk was transferred to sterile 10 ml centrifuge tubes and centrifuged at 2000 rpm for 15 mins at 4° C. Milk was aspirated from under the solid fat layer using disposable pipette and placed in fresh 10 ml tube. The pipette was carefully plunged through the fat layer into the milk layer below. 2 mg/ml Rennet solution was added to the milk at the ratio of 0.4 ml rennet to 5 ml milk, tubes were shaken then incubated at 37° C. for 1 to 2 hours. Tubes were centrifuged at 5000 rpm×15 mins at 4° C. Supernatant was removed by transfer pipette and stored at −20° C.

Antibodies in the milk were quantified by the ELISA.

### Example 13

### Collection and Storage of Blood Samples

### Materials

(i) 7 ml of 9 ml Vacutainers™ (Bectco Dickinson) for serum collection
(ii) 20 G×1.5 inch Vacutainer™ (Bectco Dickinson) needles and holder

### Method

Blood samples were taken from the jugular vein using Vacutainer™ collection tubes, holder and needle. Tubes were stored at 4° C. Samples were centrifuged at 4000 rpm×15 min at 4° C. Upper serum layer was removed using a transfer pipette and stored at −20° C.

### Example 14

### Enzyme-Linked Immunosorbent Assay

### Materials

(i) 10× Phosphate Buffer Saline (PBS); 1 L double distilled water, 1.91 g KH₂PO₄ (BDH Chemicals Aust Pty Ltd), 6.1 g Na₂HPO₄ (ASAX Chemicals), 2 g KCl (BDH Chemicals Aust Pty Ltd),
(ii) 80 g NaCl, 1.95 g NaN₃ (Sigma Aldrich), pH to 7.4
(iii) 200 ml Carbonate coating buffer pH 9.6 containing; 200 ml double distilled water, 3.18 g Na₂CO₃ (BDH), 5.88 g NaHCO₃ (BDH), 0.39 g NaN₃ (Sigma).
(iv) 0.85% saline (Excel Laboratories)
(v) 0.25 mg/ml lipase from P. fluorescens (Sigma Aldrich) in carbonate coating buffer
(vi) PBS-TW20 plate washing solution (BDH); 200 ml 10×PBS (as above), 1800 ml distilled water, 1 ml Tween-20 (Labchem)
(vii) Serum diluent; 200 ml glycerol (BDH), 29 g NaCl (BDH), 0.2 g KH₂PO₄ (BDH), 0.61 g Na₂HPO₄ (BDH), 0.2 g KCl (BDH), 1.95 g NaN₃ (Sigma), distilled water to 1 L, 1.5 ml Tween-20 (Labchem), pH to 7.4. Store 4° C. Dessicated BSA (CSL) added to desired aliquot at 1% concentration, prior to use.
(viii) Saline 0.85% (Excel Laboratories)
(ix) Donkey anti-goat IgG-Horse Radish Peroxidase (HRP) (Promega)
(x) 1M H₂SO₄ (AJAX Finechem)
(xi) TMBS EIA solution (BioRad) Parts A & B
(xii) P200 pipetteman and tips
(xiii) P20 pipetteman and tips
(xiv) Nunc™ polsorb 96 well ELISA plates
(xv) BioRad™ 96 well plate spectrophotometer model 450

### Method

100 μl of lipase in carbonate buffer was added to the wells of the ELISA plate and stored at 4° C. overnight. Plates were washed 3 times with PBS-TW. 100 μl of serum diluent was added to wells for serum analysis, and 90 μl were added to wells for milk analysis. 1 μl of serum sample and 10 μl of milk sample added to the serum diluent. The mixture was created by gentle tapping the plate. The plates were stored at 4° C. overnight. The plates were washed 3 times with PBS-TW. 100 μl of a 1/2500 dilution of Donkey anti-goat IgG-HRP in 0.85% saline added to each well. The plates were incubated at room temperature for 1 hour. The plates were washed 3 times with PBS-TW. 9 parts of Part A and 1 part of Part B of TMBS were mixed in a glass Schott bottle and 100 μl was added to each well. The plates were incubated at room temperature for 10 minutes. 100 μl M H₂SO₄ was added to each well and plate read on spectrophotometer at 450 nm. A printout of the absorbance results was obtained. The absorbance values of each milk and blood sample collected from all animals were measured. Plots of absorbance values on the y-axis against time (days) on the x-axis were prepared for individual animals and the average absorbance value for each group (comprising two test subjects).

### Example 15

### Lipase Diffusion in Milk Agar Slide

(i) Prepare 1% (1 g/100 ml) agar (Oxoid Cat No L13, Basingstoke, Hampshire, England) in Phosphate Buffer Saline (pH 7.4).
(ii) Add 100 μl of whole milk (Masters, Perth, Australia) to 5 ml 1% agar.
(iii) For slide format, 2.5 ml of ‘milk agar’ is poured over the glass and allowed to set for 10 minutes (2% milk in 1% agar).
(iv) Five 1.5 mm diameter holes in the milk agar gel were prepared with an agar punch and the agar removed by vacuum,
(v) The agar film was incubated over night with lipase from P. fluorescens (Aldrich, Milwaukee, Wis., USA). 5 mg/ml of lipase was prepared in 0.85% saline.
(vi) The diffusion zone, indicative of lipid degradation of the lipase test was compared to a plate with (a) saline, (b) lipase+antibody negative serum and (c) lipase+anti-lipase positive serum.

Results are present in FIG. 13. On each milk slide, 5 wells were filled with saline (slide 1), the lipase enzyme (slide 2), lipase with an antibody negative serum (slide 3) and lipase with an anti-lipase antibody positive serum (slide 4). In slide 2, a zone of hydrolysis is visible as the lipase enzyme hydrolyses the lipids in the milk film. The negative control (saline in slide 1) confirms that the enzyme is responsible for the zone of hydrolysis. The hydrolysis activity of the lipase enzyme can be inhibited by an anti-lipase antibody, as evident in slide 4. Slide 3, which contains an antibody negative serum, confirms that it is the antibody and not other components of serum that is inhibiting the enzyme.

### Example 15

### Milk Agar Plates

(i) 1% milk in 1% agar was prepared.
(ii) 10 mls of the solution was poured into Petri dishes and allowed to set at room temperature.
(iii) Six 1.5 mm diameter holes were prepared in the agar with a punch, and removed by vacuum.
(iv) The wells were filled with 1:1 pre-bleed sera+PBS solution (well 1), 1:1 pre-bled sera+lipase solution (well 2), 2.5 mg/ml lipase (well 3), 1:1 anti-lipase sera (38 days)+PBS solutions (well 4), 1:1 anti-lipase sera (38 days)+lipase (well 5), and 0.85% PBS (well 6) (see schema on FIG. 5 for position of each well).

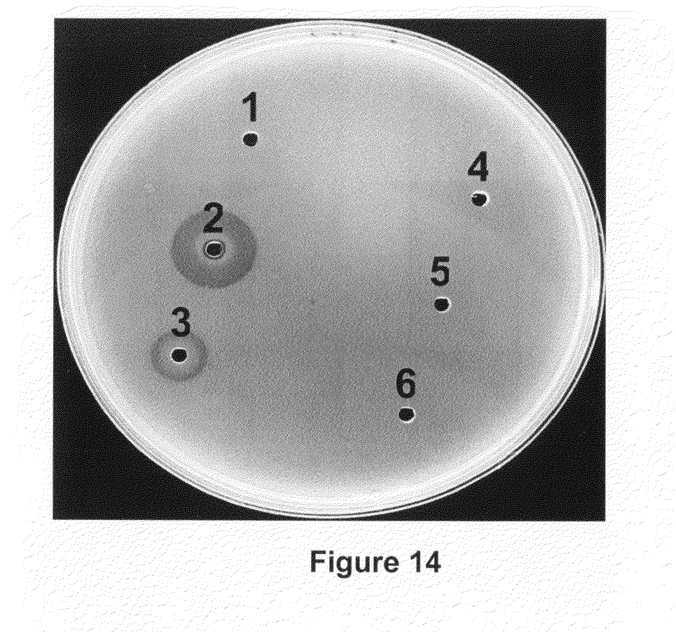

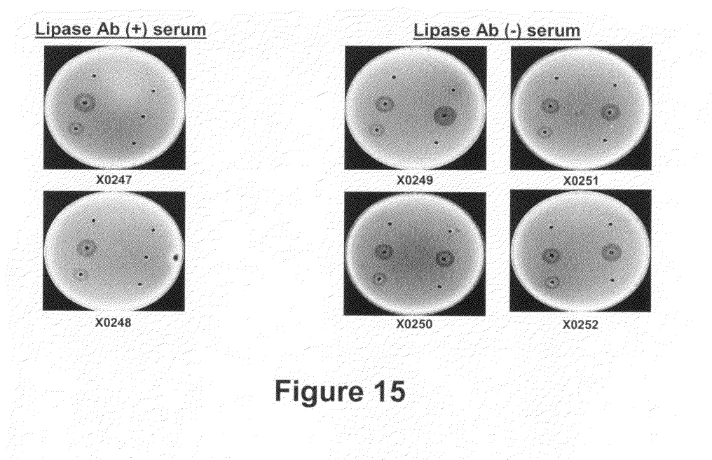

Results are present in FIGS. 14 and 15.

FIG. 14 shows a 1% milk in 1% agar plate with wells containing 1:1 ratio of pre-bleed sera+PBS solution (well 1), 1:1 pre-bled sera+lipase solution (well 2), 2.5 mg/ml lipase (well 3), 1:1 anti-lipase sera (38 days)+PBS solutions (well 4), 1:1 anti-lipase sera (38 days)+lipase (well 5), and 0.85% PBS (well 6).

In well 2 and 3, a zone of lipid hydrolysis is evident as a result of the activity of the lipase enzyme. In well 5, the activity of lipase is inhibited by the addition of serum containing anti-lipase activity. Well 1, which serum does not contain antibodies, shows no hydrolytic activity. This show that there components of the serum does not affect lipids in milk. Similarly, the serum that contains anti-lipase antibodies does not hydrolyse lipids in milk (well 4). Lane 6 is a negative control (containing only PBS) and shows no hydrolysis of lipids.

In summary, the addition of lipase hydrolyses lipids that are contained within milk contained in the milk agar plate as evident in wells 2 and 3. There appears to be elements in the serum that enhances the hydrolytic activity, when the diameter of zoning is compared between wells 3 and 2. This is not unexpected, since serum is a protein rich solution that may contain elements that are synergistic with the lipase enzyme. Nevertheless, the hydrolytic activity of the lipase enzyme, including the factors of serum, is completely inhibited by the inclusion of the anti-lipase antibody (see well 5). There was no hydrolytic activity produced by serum only (well 1 and 4) and saline (well 6).

In FIG. 15, the same milk agar plate setup was established to compare the inhibitory activity on the lipase enzyme. X0247 and X0248 contained anti-lipase antibodies from two different sources. X0249, X0250, X0251 and X0252 contains antibodies directed against other proteins. In wells 2 and 3 of each of the six plates, lipid hydrolysis was evident. In well 5 containing the respective antibodies, inhibition was only evident for milk plate X0247 and X0248 that contained the anti-lipase antibody. In contrast, the other antibodies did not inhibit lipase activity.

### Example 16

### Lipid Hydrolysis by Lipase Assay by pH

(i) In McCartney bottles, add 5 ml of 0.05M Sodium Carbonate (Ajax-Univar) to 10 ml fresh milk (Harvey Fresh, Harvey, Western Australia).
(ii) Incubate mixture at 37° C. for 5 minutes.
(iii) In a separate McCartney, incubate 500 μl of 5 mg/ml lipase at 40° C.
(iv) Add the milk/sodium carbonate solution to the lipase.
(v) Insert pH probe and record pH at desired 30 second intervals.

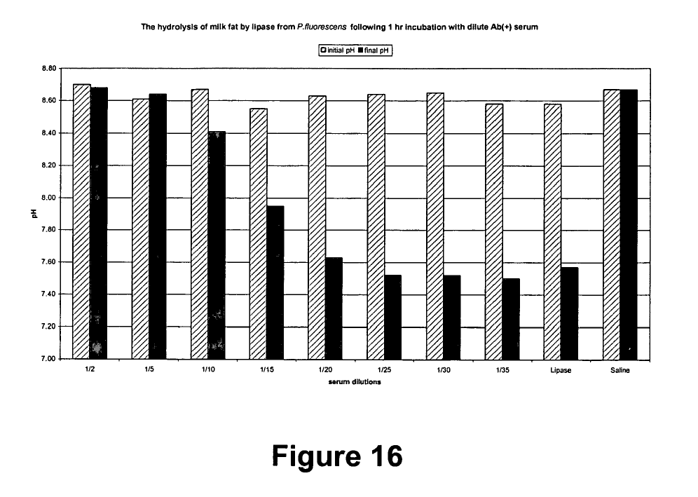

FIG. 16 summarises the results of an experiment which measures the hydrolytic activity of lipase. Briefly, fresh milk was incubated with the lipase enzyme at 37° C. The pH of milk was measured before the commencement of the experiment and at 30 second intervals after lipase was added. As lipids in the milk are hydrolysed, the free fatty acids that are liberated reduce the pH of the milk. The change in pH of milk incubated with lipase at 37° C. was plotted (FIG. 16). The reduction of pH followed a hyperbolic pattern. When anti-lipase antibodies were added, the rate of pH change was reduced in a dose-dependant manner. The rate of pH change was less when greater concentrations of the anti-lipase antibody were added. Furthermore, the shape of the curve was linear.

### Example 17

### Inhibition of Lipid Hydrolysis by Lipase Using an Anti-Lipase Antibody

(i) The lipid hydrolysis assay by pH was used in a comparative study to evaluate the inhibitory properties of anti-lipase antibodies. Specifically, a series of lipid hydrolysis assays were establish with the following additions:
   a. a pre-bleed serum (ie. antibody negative serum),
   b. 240 μl of anti-lipase positive serum,
   c. 500 μl of anti-lipase positive serum,
   d. 1000 μl of anti-lipase positive serum.
(ii) pH was measured at 30 seconds interval and the results were plotted.

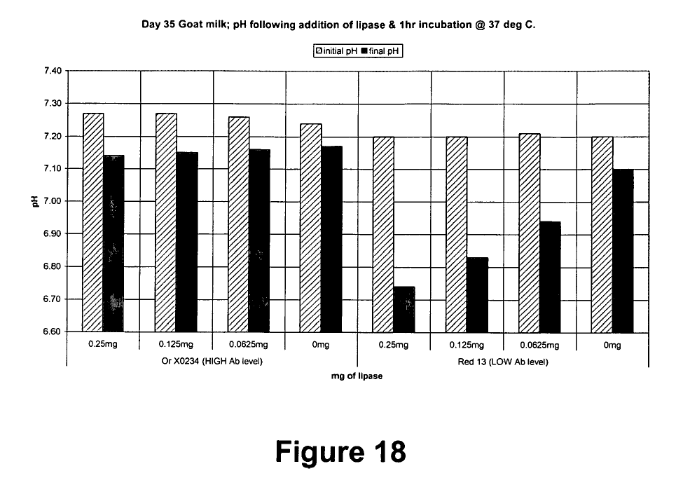

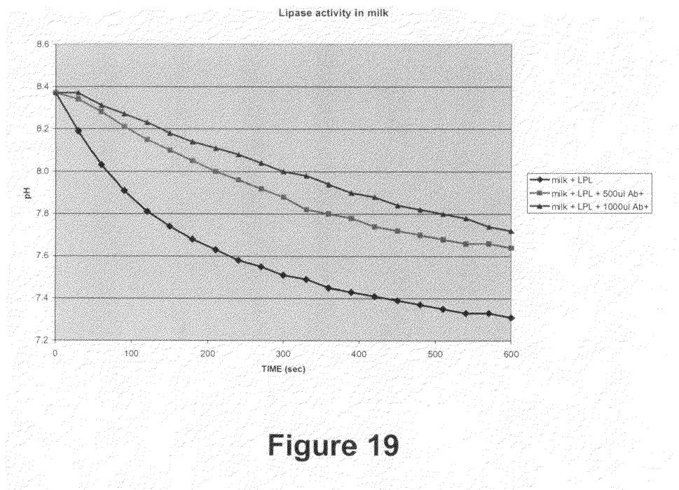

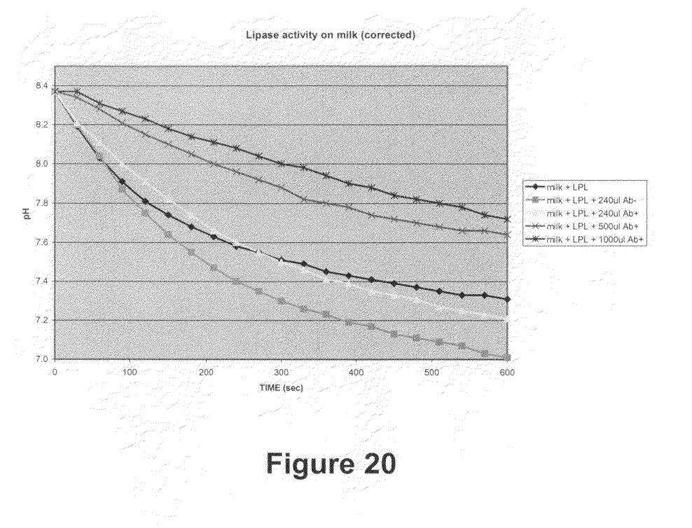

Results from examples 16 and 17 are presented in FIGS. 16, 17 and 18. Further data is presented in FIGS. 19 and 20.

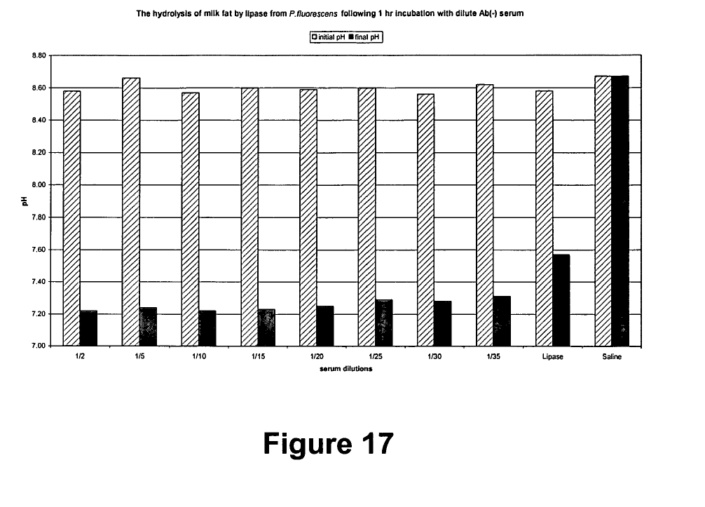

In FIG. 17, the data from FIG. 16 are plotted with two additional data sets. Milk was incubated with lipase and an antibody negative serum (▪). The curve showing pH change was similar to the curve for the control (ie. milk+lipase only). The anti-lipase antibody was titrated to lower concentrations (ie, 1000 μl, 500 μl and 250 μl). The results suggest that antibody concentration is rate-limiting factor. The 250 μl curve is parabolic an not linear, suggesting that antibodies level are limited and hydrolytic activity of the enzyme is not complete.

The experiment was repeated at 10° C. and 4° C. At 10° C., the rate of pH change between lipase spike milk that contain antibody and those that do not contain the antibody were similar, indicating that mechanisms other that lipase hydrolysis is at work. At 4° C., the rate of change without antibody was greater when compared to the sample containing the anti-lipase antibody.

### Example 18

### Shelf-Life Assessment by Measuring pH as an Indicator of Lipid Hydrolysis

(i) Hydrolysis of lipids as measured by pH change was performed at 10° C. and at 4° C.
(ii) pH was measured daily from a series of samples containing saline (PBS), 10% pre-bleed serum (antibody negative serum) and 10% anti-lipase positive serum.

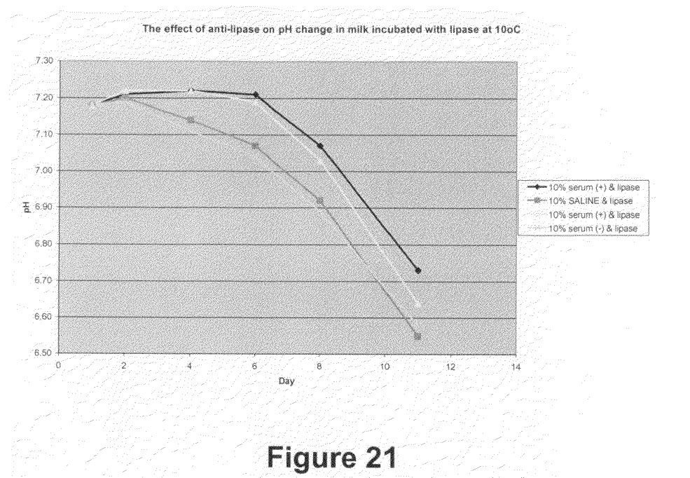

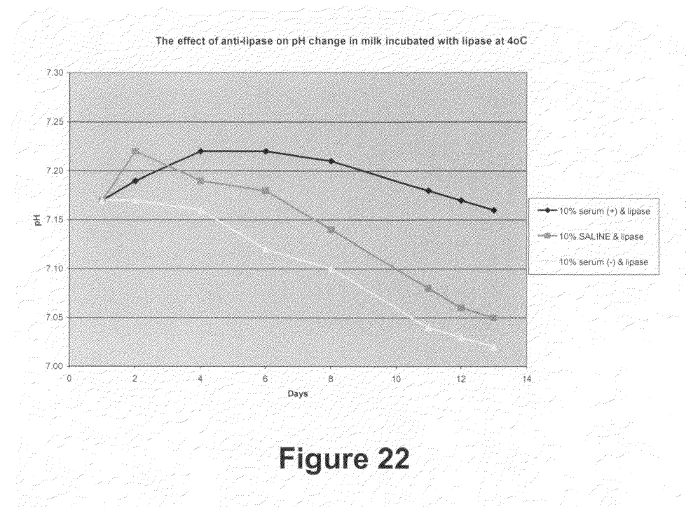

Results are presented in FIGS. 21 and 22.

Milk was incubated with lipase and eight dilutions of serum containing anti-lipase antibody. A positive control (lipase only with no serum) and a negative serum (saline or PBS only) were also prepared for comparison. The pH of milk was measured prior to start of the experiment and one hour after incubation. At the 1:2 and 1:5 dilutions, the pH change was minimal. As the concentrations of antibodies are decreased, the pH changes are distinct, suggesting an antibody dose-effect on the lipolysis of milk fat by lipase.

In contrast, milk that was incubated with serum containing no antibodies showed the hydrolytic activity of lipase. The change in pH over the incubation period (1 hr) suggests that lipase is hydrolyzing the lipids in the milk.

Although the invention has been described with reference to certain preferred embodiments, it will be appreciated that many variations and modifications may be made within the scope of the broad principles of the invention. Hence, it is intended that the preferred embodiments and all of such variations and modifications be included within the scope and spirit of the invention.

1. A method for improving the half-life of milk comprising the step of: a) contacting milk with an antibody raised against at least one of the following: i) a molecule required for survival by at least a micro-organism that is responsible for reducing the half-life of milk; ii) a molecule required for growth by at least a micro-organism that is responsible for reducing the half-life of milk; iii) a molecule required for survival by at least a micro-organism that aids other micro-organisms to reduce the half-life of milk; iv) a molecule required for growth by at least a micro-organism that aids other micro-organisms to reduce the half-life of milk.

2. The method of claim 1 wherein the method prolongs the shelf-life of the milk.

3. The method of claim 1 wherein the antibody, when contacted with the milk, forms an antibody-antigen interaction with the molecule which depletes, removes or prevents the molecule from being a nutrient source for the microorganism or prevents the activity of the molecule.

4. The method of claim 3 wherein the antibody is an antibody generated against lipoprotein lipase from Pseudomonas fluorescens.

5. The method of any one of claims 1 wherein the antibody molecules are chosen from the list comprising: intact immunoglobulin molecules, substantially intact immunoglobulin molecules and portions of an immunoglobulin molecule that contain the paratope.

6. The method of claim 5 wherein the portion of an immunoglobulin molecule that contain the paratope is selected from the list comprising: Fab, Fab′, F(ab′)2 and F(v) portions.

7. A method for improving the half-life of milk comprising the step of: a) inducing the production of at least an antibody in the mammary gland of an animal, which antibody is raised against at least one of the following: i) a molecule required for survival by at least a micro-organism that is responsible for reducing the half-life of milk; ii) a molecule required for growth by at least a micro-organism that is responsible for reducing the half-life of milk; iii) a molecule required for survival by at least a micro-organism that aids other micro-organisms to reduce the half-life of milk; iv) a molecule required for growth by at least a micro-organism that aids other micro-organisms to reduce the half-life of milk.

8. A method according to claim 7 wherein the method prolongs the shelf-life of the milk.

9. A method for the induction of antibody production in milk for improving the half-life of milk comprising the step of: a) implanting at least one antigen releasing device adjacent to or within at least one supramammary lymph node, wherein in use the antigen releasing device releases an antigen into the tissue area around the supramammary lymph node which stimulates antibody secretion into a mammary gland.

10. The method of claim 9 wherein the shelf-life of the milk is prolonged.

11. The method of claim 9 wherein the implant is located in sufficient proximity to the supramammary gland such that the release of antigen from the antigen releasing device induces the production of antibodies in milk over the life of the antigen releasing device.

12. The method of claim 11 wherein the antigen releasing device is implanted at a distance of up to 100 mm from at least one supramammary lymph node.

13. The method of claim 12 wherein the antigen releasing device is implanted at a distance of between about 1 mm and 100 mm from at least one supramammary lymph node.

14. The method of claim 13 wherein the antigen releasing device is implanted at a distance of between about 50 mm and 100 mm from at least one supramammary lymph node.

15. A method for inducing the sustained release of antibodies in milk to prolong the half-life of the milk comprising the steps of: a) administering a primer composition; and b) implanting at least one antigen releasing device adjacent to, within close proximity of or within at least one supramammary lymph node, wherein in use the antigen releasing device releases an antigen into the tissue area around the supramammary lymph node which stimulates antibody secretion into a mammary gland.

16. A method for inducing the sustained release of antibodies in milk to prolong the half-life of the milk comprising the step of: a) implanting at least one antigen releasing device adjacent to, within close proximity of or within at least one supramammary lymph node, and b) administering a booster composition comprising antigen to a mammal after the antigen releasing device has been implanted wherein in use the antigen releasing device releases an antigen into the tissue area around the supramammary lymph node which stimulates antibody secretion into a mammary gland.

17. A method for inducing the sustained release of antibodies in milk to prolong the half-life of the milk comprising the step of: a) administering a primer composition; b) implanting at least one antigen releasing device adjacent to, within close proximity of or within at least one supramammary lymph node; and c) administering a booster composition comprising antigen to a mammal after the antigen releasing device has been implanted wherein in use the antigen releasing device releases an antigen into the tissue area around the supramammary lymph node which stimulates antibody secretion into a mammary gland.

18. The method of claim 15 or 17 wherein the primer is administered by an administration route selected from: intramammary, intraperitoneal, intramuscular or intranasal.

19. The method of claim 15 or 17 wherein administration of a primer takes place before implanting the antigen releasing device.

20. The method of claim 15 or 17 wherein administration of a primer takes place during implantation of the antigen releasing device.

21. The method of claim 15 or 17 where the primer composition is delivered to a mucosal surface.

22. The method of claim 15 or 17 wherein the primer composition is administered in a single administration.

23. The method of claim 15 wherein the primer composition is administered in a number of administrations at intervals over a period of days or weeks.

24. The method of claim 16 or 17 wherein the booster is administered by an administration route selected from: intramammary, intraperitoneal, intramuscular and/or intranasal.

25. A method according to claim 16 wherein the booster composition is delivered to a mucosal surface.

26. The method of claim 16 wherein the booster composition is administered in a single administration.

27. The method of claim 16 wherein the booster composition is administered in a number of administrations at intervals over a period of days or weeks.

28. The method of claim 15 wherein the antigen administered is the same for each step of the method.

29. The method of claim 15 wherein the antigen releasing device, the priming composition and the booster composition further comprise an adjuvant.

30. A method according to claim 1 further comprising a preselection step prior to administration of the antigen releasing device wherein animals showing the best antibody titre responses are used in the methods of any one of claims 1 to 29.

31. A method for the production of milk with an improved half-life containing antibodies, comprising the steps of: a) inducing antibodies using the method of claim; and b) collecting the antibody containing milk from the mammal wherein the antibodies prolong the half-life of the milk.

32. The method of claim 31 wherein the antibodies prolong the shelf-life of the milk.

33. A milk product with prolonged half-life, said milk product being prepared from milk generated according to the method of claim 1 and wherein milk produced by the method is processed into the milk product.

34. A milk product with prolonged shelf-life, said milk product being prepared from milk generated according to the method of claim 1 and wherein milk produced by the method is processed into the milk product.

35. A milk product with prolonged half-life, said product being a milk product or derivative whose half-life is prolonged by the addition of an antibody raised against at least one of the following: i) a molecule required for survival by at least a micro-organism that is responsible for reducing the half-life of milk; ii) a molecule required for growth by at least a micro-organism that is responsible for reducing the half-life of milk; iii) a molecule required for survival by at least a micro-organism that aids other micro-organisms to reduce the half-life of milk; iv) a molecule required for growth by at least a micro-organism that aids other micro-organisms to reduce the half-life of milk.

36. A milk product with prolonged shelf-life, said product being a milk product or derivative whose shelf-life is prolonged by the addition of an antibody raised against at least one of the following: i) a molecule required for survival by at least a micro-organism that is responsible for reducing the shelf-life of milk; ii) a molecule required for growth by at least a micro-organism that is responsible for reducing the shelf-life of milk; iii) a molecule required for survival by at least a micro-organism that aids other micro-organisms to reduce the shelf-life of milk; iv) a molecule required for growth by at least a micro-organism that aids other micro-organisms to reduce the shelf-life of milk.

37. The milk product of claim 33 wherein milk containing antibodies is subjected to heat treatment, ultra violet radiation, concentration, supplementation with food additives, drying into a concentrate or milk powder to form the milk product.
